(19) United States
(12) Patent Application Publication
(10) Pub. No.: US 2025/0171395 A1
(43) Pub. Date: May 29, 2025

(54) PROCESS FOR THE PREPARATION OF AMPHETAMINE AND DERIVATIVES THEREOF

(71) Applicant: Granules India Limited, Hyderabad (IN)

(72) Inventors: Venkata Srinivas Pullela, Hyderabad (IN); Sampath Kumar Upparapalli, Hyderabad (IN); Venkateswara Rao Kalapala, Hyderabad (IN)

(73) Assignee: Granules India Limited, Hyderabad (IN)

(21) Appl. No.: 18/962,271

(22) Filed: Nov. 27, 2024

(30) Foreign Application Priority Data
Nov. 28, 2023 (IN) 202341080644

(51) Int. Cl. C07C 209/74; B01J 21/18; B01J 23/44; B01J 35/00; C07C 231/12

(52) U.S. Cl. CPC C07C 209/74; B01J 21/18; B01J 23/44; B01J 35/19; C07C 231/12

(57) ABSTRACT

The present invention relates to an improved process for the preparation of amphetamine and it's derivatives thereof, using a silane reagent as a hydrogen source, which is economical, green and safe method with simple operation, mild conditions and high product purity.

### FIELD OF INVENTION

The present invention relates to an improved process for the preparation of amphetamine and it's derivatives thereof.

### BACKGROUND OF THE INVENTION

The compound 1-Phenyl-2-aminopropane generally named as amphetamine, has the following chemical structure:

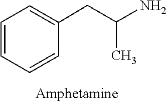

Lisdexamfetamine is an amphetamine prodrug, comprising an 1-lysine amino acid covalently bonded to dextroamphetamine (d-amphetamine). Lisdexamfetamine is approved and marketed under the brand name of Vyvanse in the United States for the treatment of attention-deficit hyperactivity disorder in adults and pediatric patients 6 years and older.

Lisdexamfetamine chemically known as (2S)-2,6-diamino-N-[(1S)-1-methyl-2-phenylethyl]hexanamide and has the following structural formula:

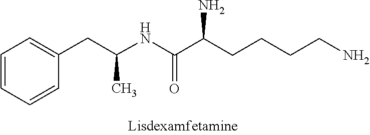 [SMILES: C[C@@H](Cc1ccccc1)NC(=O)[C@@H](N)CCCCN]

The U.S. Pat. No. 6,399,828 patent describes two prior art methods for synthesizing amphetamine from norephedrine. In one method, iodination of norephedrine hydrochloride (HI, P₄) and reduction to amphetamine. In the other method, norephedrine hydrochloride is chlorinated using thionyl chloride and then the chlorinated product, following recrystallization from a methanol-isopropyl ether solution, is subjected to catalytic hydrogenation over palladium on carbon and hydrogen to amphetamine.

The U.S. Pat. No. 6,399,828 patent also discloses a preparation method of amphetamine comprising acetylation of norephedrine hydrochloride with acetic anhydride in acetic acid to acetyl norephedrine, is subjected to catalytic hydrogenation over palladium on carbon and hydrogen to amphetamine.

The U.S. Pat. No. 7,705,184 patent discloses a method for preparing amphetamine comprising, dissolving a crude chlorinated phenylpropanolamine hydrochloride in water to form an aqueous solution, contacting the aqueous solution with carbon, and hydrogenating the carbon-treated aqueous solution over palladium on carbon and hydrogen to amphetamine.

Similarly, the U.S. Pat. No. 8,614,346 patent discloses a method for preparing Lisdexamfetamine comprises, catalytic hydrogenation of halo-Lisdexamfetamine over palladium on carbon and hydrogen to get Lisdexamfetamine.

The above prior-art methods involve hydrogenation reaction using hydrogen is a dangerous process under national key supervision (National Key Supervision Hazardous Process Catalog 2013 Edition). Because the hydrogenation process has the following dangerous characteristics:
1) The reaction materials have a risk of explosion, the explosion limit of hydrogen is 4% to 75%, and it has high risk of explosion;
2) Hydrogenation is a strong exothermic reaction. When hydrogen comes into contact with steel at high temperatures, the carbon molecules in the steel are prone to react with hydrogen to form hydrocarbons, which reduces the strength of steel equipment and causes hydrogen embrittlement;
3) During the catalyst regeneration and activation process, it is easy to cause explosions;
4) There are incompletely reacted hydrogen and other impurities in the hydrogenation reaction tail gas, which can easily cause fires and explosions when discharged.

Hence, there is a need in the art for an improved process for the preparation of amphetamine and derivatives thereof, which is economical, green and safe method with simple operation, mild conditions and high product purity. Therefore, it is particularly to develop a green, economical, and cost-controllable production process for amphetamine and derivatives thereof, to meet the challenges of increasing market demand and the company's core competitiveness.

### OBJECTIVES OF THE INVENTION

It is an object of the present invention to overcome the shortcomings of the prior art.

It is an object of the present invention to provide an improved process for the preparation of amphetamine and derivatives thereof, which is economical, green and safe method with simple operation, mild conditions and high product purity.

It is another object of the present invention to provides an improved process for the preparation of amphetamine and derivatives thereof, using silane reagent as hydrogen source, which having strong operational tolerance and is easy to control in industrial production.

### Advantages of the Invention

The usage of silane reagent in the present invention can avoid the usage of hydrogen gas and pressure reaction vessels and other operations involved in the reported process.

The usage of silane reagent in the present invention, the reaction can be completed at room temperature and pressure, which can greatly avoid the supervision caused by hydrogenation and cryogenic processes.

The usage of silane reagent in the present invention, there is no need to avoid the influence of air, moisture and other factors. It has strong operational tolerance and is easy to control in industrial production.

The usage of silane reagent in the present invention, the yield and quality are superior to the catalytic hydrogenation preparation process, which can significantly reduce production costs.

The usage of silane reagent in the present invention, the reaction can be quantitative, avoiding the potential rear emissions and safety hazards of a large amount of waste gas when using hydrogen.

### SUMMARY OF THE INVENTION

According to an aspect of the present invention, there is provided an improved process for the preparation of amphetamine and derivatives thereof, which is economical, green and safe method with simple operation, mild conditions and high product purity.

According to one aspect of the present invention, there is provided an improved process for the preparation of amphetamine compound of general formula I or isomer thereof:

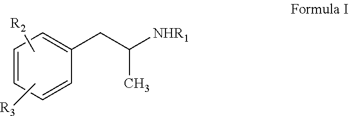

wherein R₁ is hydrogen or a lower alkyl group, and R₂ and R₃ are independently selected from hydrogen, halogen, lower alkyl groups, lower alkoxy groups, lower alkyl groups substituted with 1 to 5 halogens, lower alkoxy groups substituted with 1 to 5 halogens;
comprising reacting compound of general formula III or isomer thereof or its salt:

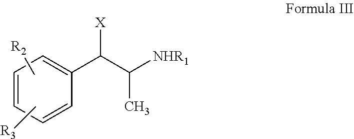

wherein X is halogen, hydroxy, hydroxy protecting group; R₁, R₂ and R₃ have the same meanings as identified above;
with a silane reagent and a catalyst in presence of a suitable solvent to provide amphetamine compound of general formula I or isomer thereof.

According to another aspect of the present invention, there is provided reacting compound of general formula III or isomer or its derivatives thereof or its salt with a silane reagent as a hydrogen source to produce amphetamine compound of general formula I or isomer thereof or its derivatives thereof like lisdexamphetamine.

According to another aspect of the present invention, there is provided an improved process for the preparation of acyl-amphetamine compound of general formula II or isomer thereof:

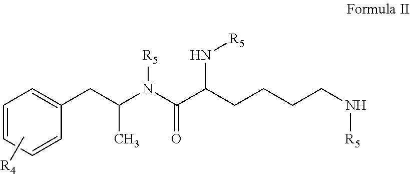

wherein R₄ is hydrogen, halogen, C₁-C₆ alkyl, or C₁-C₆ alkoxy; R₅ represents independently for each occurrence hydrogen or C₁-C₆ alkyl; comprising:
a) reacting acyl-halamphetamine compound of general formula IV or isomer thereof:

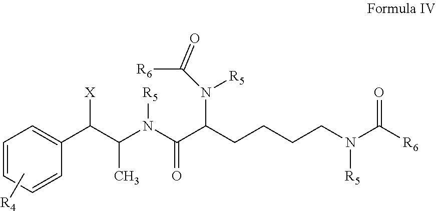

wherein X is halogen, hydroxy, hydroxy protecting group; R₄ and R₅ have the same meanings as identified above; R₆ represents independently for each occurrence C₁-C₆ alkyl, C₁-C₆ haloalkyl, C₁-C₆ alkoxy, or aryl;
with a silane reagent and a catalyst in presence of a suitable solvent to provide acyl-amphetamine compound of general formula V or isomer thereof; and

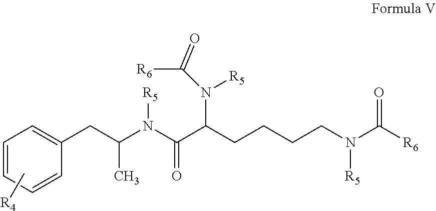

wherein R₄, R₅ and R₆ have the same meanings as identified above;
b) deprotecting the acyl-amphetamine compound of general formula V or isomer thereof with a suitable deprotecting agent to provide acyl-amphetamine compound of general formula II or isomer thereof.

According to yet another aspect of the present invention, there is provided an improved process for the preparation of acyl-amphetamine compound of general formula II or isomer thereof:

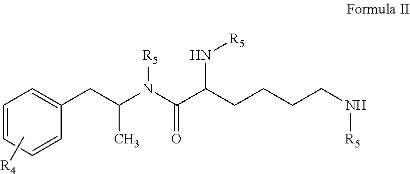

wherein R₄ is hydrogen, halogen, C₁-C₆ alkyl, or C₁-C₆ alkoxy; R₅ represents independently for each occurrence hydrogen or C₁-C₆ alkyl; comprising:
a) deprotecting acyl-halamphetamine compound of general formula IV or isomer thereof:

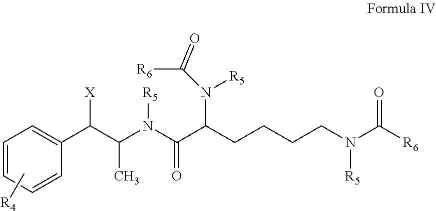

wherein X is halogen, hydroxy, hydroxy protecting group; R₄ and R₅ have the same meanings as identified above; R₆ represents independently for each occurrence C₁-C₆ alkyl, C₁-C₆ haloalkyl, C₁-C₆ alkoxy, or aryl;
with a suitable deprotecting agent to provide acyl-halamphetamine compound of general formula VI or isomer thereof; and

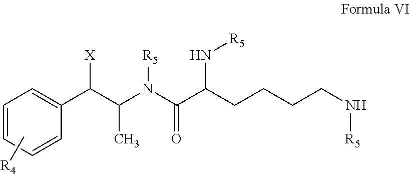

wherein X, R₄ and R₅ have the same meanings as identified above;
b) reacting the acyl-halamphetamine compound of general formula VI or isomer thereof with a silane reagent and a catalyst in presence of a suitable solvent to provide acyl-amphetamine compound of general formula II or isomer thereof.

### DETAILED DESCRIPTION

The following description with accompanying drawings is provided to assist in a comprehensive understanding of exemplary embodiments of the invention. It includes various specific details to assist in that understanding but these are to be regarded as merely exemplary.

Accordingly, those of ordinary skill in the art will recognize that various changes and modifications of the embodiments described herein can be made without departing from the scope of the invention. In addition, descriptions of well-known functions and constructions are omitted for clarity and conciseness.

The terms and words used in the following description and claims are not limited to the bibliographical meanings, but, are merely used by the inventor to enable a clear and consistent understanding of the invention. Accordingly, it should be apparent to those skilled in the art that the following description of exemplary embodiments of the present invention are provided for illustration purpose only and not for the purpose of limiting the scope of the invention as defined by the appended claims and their equivalents.

It is to be understood that the singular forms “a,” “an,” and “the” include plural referents unless the context clearly dictates otherwise.

Features that are described and/or illustrated with respect to one embodiment may be used in the same way or in a similar way in one or more other embodiments and/or in combination with or instead of the features of the other embodiments.

It should be emphasized that the term “comprises/comprising” when used in this specification is taken to specify the presence of stated features, steps or components but does not preclude the presence or addition of one or more other features, steps, components or groups thereof.

The term “solvent/suitable solvent” used in the present invention is selected from the group comprising of water, alcohols, ethers, amides, esters, nitriles, sulfoxides, ketones, hydrocarbons and halogenated hydrocarbons; wherein alcohol is selected from the group consisting of methanol (MeOH), ethanol (EtOH), iso-propanol (IPA), n-butanol, iso-butanol and the like; ester is selected from the group consisting of ethyl acetate, isopropyl acetate (IPAc); ketone is selected from the group consisting of acetone, methyl isobutyl ketone, methyl ethyl ketone; ether is selected from the group consisting of methyl tert-butyl ether, diisopropyl ether, diethyl ether tetrahydrofuran, 2-methyl tetrahydrofuran, cyclopentyl methyl ether, dioxane and the like; halogenated solvent is selected from the group consisting of methylene dichloride (MDC), chloroform, chlorobenzene, bromobenzene and the like; hydrocarbons is selected from the group consisting of heptane, hexane, cyclohexane, cycloheptane, toluene, xylene, cyclohexane and the like; nitrile is selected from the group consisting of acetonitrile (ACN), propionitrile and the like; amide is selected from the group consisting of N,N-dimethylformamide (DMF), N,N-dimethyl acetamide (DMAc) and the like; sulfoxide such as dimethyl sulfoxide; sulfone; or mixtures thereof.

The term “silane reagent” used in the present invention until unless specified is selected form the group consisting of R₁SiOSiR₂ or RiSiR₂R₃; wherein R₁, R₂ and R₃ are independently selected from hydrogen, halogen, hydroxy, substituted or unsubstituted lower alkyl groups, substituted or unsubstituted lower alkoxy groups, and substituted or unsubstituted aryl groups, polymethylhydrosilane (PMHS), polydimethylsiloxane (PDMS) and the like.

The term “catalyst” used in the present invention until unless specified is selected from the group consisting of palladium hydroxide, palladium on carbon, platinum on carbon, Fe, Raney nickel, platinum oxide, zinc and the like.

The term “hydroxy protecting group” can be selected from but not limited to methyl, ethyl, acetate, ethylacetate, propionate, ethylene glycol, propylene glycol, 4-methoxybenzyl, benzyl, trityl, trimethylsilyl, tetrahydropyranyl, and benzoyl and the like.

The term “suitable deprotecting agent” can be selected from but not limited to hydrochloric acid, hydrobromic acid, sulfuric acid, methane sulfonic acid (MSA), p-toluene sulfonic acid, acetic acid, trifluoroacetic acid, lithium hydroxide, sodium hydroxide (NaOH), potassium hydroxide and the like.

In one embodiment of the present invention provides an improved process for the preparation of amphetamine compound of general formula I or isomer thereof:

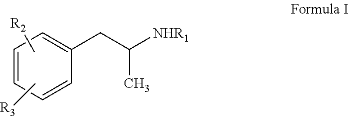

wherein R₁ is hydrogen or a lower alkyl group, and R₂ and R₃ are independently selected from hydrogen, halogen, lower alkyl groups, lower alkoxy groups, lower alkyl groups substituted with 1 to 5 halogens, lower alkoxy groups substituted with 1 to 5 halogens;
comprising reacting compound of general formula III or isomer thereof or its salt,

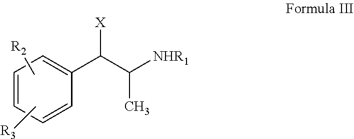

wherein X is halogen, hydroxy, hydroxy protecting group; R₁, R₂ and R₃ have the same meanings as identified above;
with a silane reagent and a catalyst in presence of a suitable solvent to provide amphetamine compound of general formula I or isomer thereof.

The starting compound of general formula III or isomer thereof or salt thereof, can be prepared by the methods known in art or by the process described herein the present invention.

The aforementioned process involves the reaction of compound of general formula III or isomer thereof or its salt such as hydrochloride, nitrate, sulfate and the like; preferably hydrochloride, wherein X is halogen selected from Cl, Br, I and F, hydroxy, hydroxy protecting group; preferably Cl; R₁ is hydrogen or a lower alkyl group; preferably hydrogen, and R₂ and R₃ are independently selected from hydrogen, halogen, lower alkyl groups, lower alkoxy groups, lower alkyl groups substituted with 1 to 5 halogens, lower alkoxy groups substituted with 1 to 5 halogens; preferably hydrogen; with a silane reagent and a catalyst in presence of a suitable solvent under appropriate reaction conditions to provide amphetamine compound of general formula I or isomer thereof.

The silane reagent, catalyst and a suitable solvent used in the process is as defined above but also selected from triethylsilane, tetramethyldisiloxane, trichlorosilane, trimethylsilanol, phenylsilane, diphenylsilane, diphenylchlorosilane, tetraphenyldisilane and the like; preferably triethylsilane and tetramethyldisiloxane; the catalyst is selected from palladium hydroxide, palladium on carbon, platinum on carbon, Raney nickel, platinum oxide and the like; preferably palladium on carbon; and the suitable solvent is selected from water or alcohols such as methanol, ethanol, iso-propanol, n-butanol, iso-butanol and the like; preferably water.

The said reaction is carried out at a suitable temperature of about 0° C. to about 60° C. for sufficient period of time; preferably for about 4 to 5 hours at about 5° C. to about 35° C.

The amphetamine compound of general formula I or isomer thereof obtained in the present invention can be optionally isolated.

In another embodiment of the present invention provides an improved process for the preparation of amphetamine compound of general formula I or isomer thereof as shown in the below scheme:

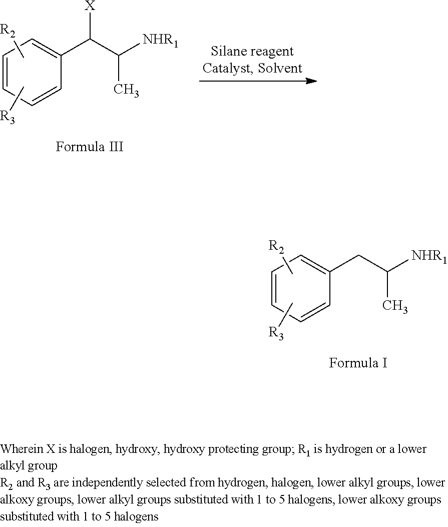

In another embodiment of the present invention provides reacting compound of general formula III or isomer thereof or its derivative thereof or its salt with a silane reagent as a hydrogen source to produce amphetamine compound of general formula I or isomer thereof or its derivatives thereof.

The amphetamine compound of general formula I or isomer thereof or its derivatives thereof obtained in the present invention is selected from but not limited to Amphetamine, Dexamfetamine, Levoamphetamine, Lisdexamfetamine and the like.

In one embodiment, the present invention provides an improved process for the preparation of Lisdexamphetamine comprising reacting compound of general formula III or isomer thereof or its derivative thereof or its salt with a silane reagent as a hydrogen source and further converting it to Lisdexamphetamine according to the methods known in art or by the process described herein the present invention.

In another embodiment of the present invention provides an improved process for the preparation of acyl-amphetamine compound of general formula II or isomer thereof:

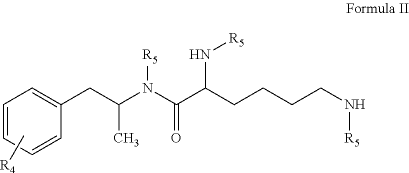

wherein R₄ is hydrogen, halogen, C₁-C₆ alkyl, or C₁-C₆ alkoxy; R₅ represents independently for each occurrence hydrogen or C₁-C₆ alkyl; comprising:
a) reacting acyl-halamphetamine compound of general formula IV or isomer thereof:

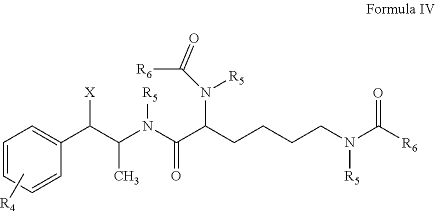

wherein X is halogen, hydroxy, hydroxy protecting group; R₄ and R₅ have the same meanings as identified above; R₆ represents independently for each occurrence C₁-C₆ alkyl, C₁-C₆ haloalkyl, C₁-C₆ alkoxy, or aryl;
with a silane reagent and a catalyst in presence of a suitable solvent to provide acyl-amphetamine compound of general formula V or isomer thereof; and

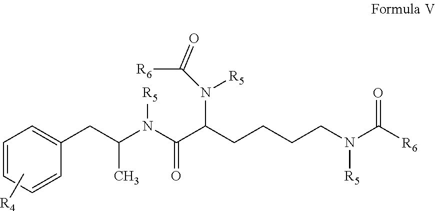

wherein R₄, R₅ and R₆ have the same meanings as identified above;
b) deprotecting the acyl-amphetamine compound of general formula V or isomer thereof with a suitable deprotecting agent to provide acyl-amphetamine compound of general formula II or isomer thereof.

The starting acyl-halamphetamine compound of general formula IV or isomer thereof can be prepared by the methods known in art or by the process described herein the present invention.

The step a) of aforementioned process involves the reaction of acyl-halamphetamine compound of general formula IV or isomer thereof, wherein X is halogen selected from Cl, Br, I and F, hydroxy, hydroxy protecting group; preferably Cl; R₄ is hydrogen, halogen, C₁-C₆ alkyl, or C₁-C₆ alkoxy; preferably hydrogen; R₅ represents independently for each occurrence hydrogen or C₁-C₆ alkyl; preferably hydrogen; and R₆ represents independently for each occurrence C₁-C₆ alkyl, C₁-C₆ haloalkyl, C₁-C₆ alkoxy, or aryl; preferably C₁-C₆ haloalkyl such as —CF₃ or C₁-C₆ alkoxy such as —O(CH₃)₃; with a silane reagent and a catalyst in presence of a suitable solvent under appropriate reaction conditions to provide acyl-amphetamine compound of general formula V or isomer thereof.

The silane reagent, catalyst and a suitable solvent used in the process is as defined above but also selected from triethylsilane, tetramethyldisiloxane (TMDS), trichlorosilane, trimethylsilanol, phenylsilane, diphenylsilane, diphenylchlorosilane, tetraphenyldisilane and the like; preferably triethylsilane and tetramethyldisiloxane; the catalyst is selected from palladium hydroxide, palladium on carbon, platinum on carbon, Raney nickel, platinum oxide and the like; preferably palladium on carbon; and the suitable solvent is selected from water or alcohols such as methanol, ethanol, iso-propanol, n-butanol, iso-butanol and the like; preferably water.

The said reaction is carried out at a suitable temperature of about 0° C. to about 60° C. for sufficient period of time; preferably for about 4 to 5 hours at about 5° C. to about 35° C.

The step b) of aforementioned process involves the deprotection of acyl-amphetamine compound of general formula V or isomer thereof, wherein R₄, R₅ and R₆ have the same meanings as identified above step a); with a suitable deprotecting agent under appropriate reaction conditions to provide acyl-amphetamine compound of general formula II or isomer thereof.

The suitable deprotecting agent used in the process is as defined above but also selected from methane sulfonic acid, sulfuric acid, sodium hydroxide, potassium hydroxide and the like; preferably methane sulfonic acid and sodium hydroxide.

The said reaction is carried out in presence of a suitable solvent selected from water or alcohols such as methanol, ethanol, iso-propanol, n-butanol, iso-butanol and the like; preferably water, methanol or iso-propanol.

The said reaction is carried out at a suitable temperature of about 0° C. to about 60° C. for sufficient period of time; preferably for about 4 to 5 hours at about 25° C. to about 55° C.

In another embodiment of the present invention provides an improved process for the preparation of acyl-amphetamine compound of general formula II or isomer thereof as shown in the below scheme:

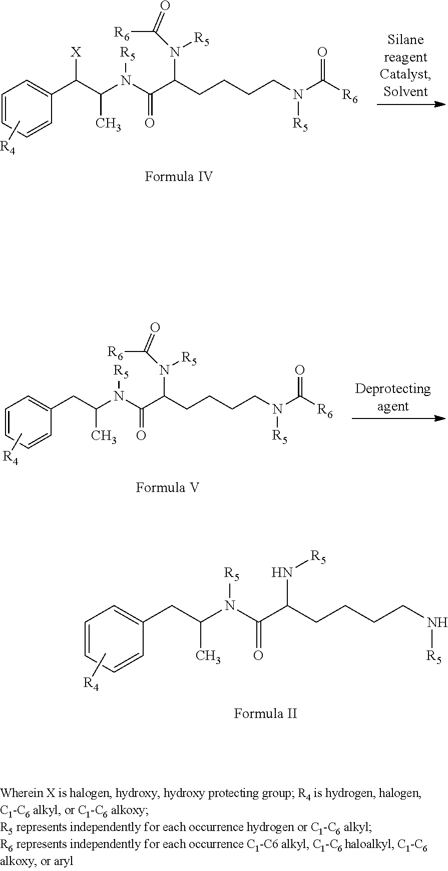

In another aspect of the present invention provides an improved process for the preparation of acyl-amphetamine compound of general formula II or isomer thereof:

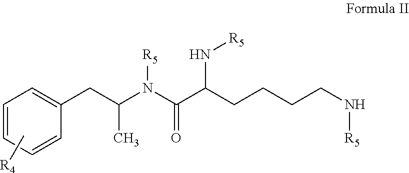

wherein R₄ is hydrogen, halogen, C₁-C₆ alkyl, or C₁-C₆ alkoxy; R₅ represents independently for each occurrence hydrogen or C₁-C₆ alkyl; comprising:
a) deprotecting acyl-halamphetamine compound of general formula IV or isomer thereof:

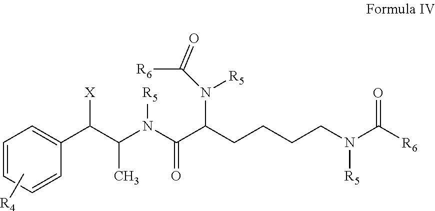

wherein X is halogen, hydroxy, hydroxy protecting group; R₄ and R₅ have the same meanings as identified above; R₆ represents independently for each occurrence C₁-C₆ alkyl, C₁-C₆ haloalkyl, C₁-C₆ alkoxy, or aryl;
with a suitable deprotecting agent to provide acyl-halamphetamine compound of general formula VI or isomer thereof; and

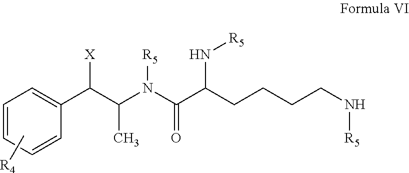

wherein X, R₄ and R₅ have the same meanings as identified above;
b) reacting the acyl-halamphetamine compound of general formula VI or isomer thereof with a silane reagent and a catalyst in presence of a suitable solvent to provide acyl-amphetamine compound of general formula II or isomer thereof.

The starting acyl-halamphetamine compound of general formula IV or isomer thereof can be prepared by the methods known in art or by the process described herein the present invention.

The step a) of aforementioned process involves the deprotection of acyl-halamphetamine compound of general formula IV or isomer thereof, wherein X is halogen selected from Cl, Br, I and F, hydroxy, hydroxy protecting group; preferably Cl; R₄ is hydrogen, halogen, C₁-C₆ alkyl, or C₁-C₆ alkoxy; preferably hydrogen; R₅ represents independently for each occurrence hydrogen or C₁-C₆ alkyl; preferably hydrogen; and R₆ represents independently for each occurrence C₁-C₆ alkyl, C₁-C₆ haloalkyl, C₁-C₆ alkoxy, or aryl; preferably C₁-C₆ haloalkyl such as —CF₃ or C₁-C₆ alkoxy such as —O(CH₃)₃; with a suitable deprotecting agent under appropriate reaction conditions to provide acyl-halamphetamine compound of general formula VI or isomer thereof.

The suitable deprotecting agent used in the process is as defined above but also selected from methane sulfonic acid, sulfuric acid, sodium hydroxide, potassium hydroxide and the like; preferably methane sulfonic acid and sodium hydroxide.

The said reaction is carried out in presence of a suitable solvent selected from water or alcohols such as methanol, ethanol, iso-propanol, n-butanol, iso-butanol and the like; preferably water, methanol or iso-propanol.

The said reaction is carried out at a suitable temperature of about 0° C. to about 60° C. for sufficient period of time; preferably for about 4 to 5 hours at about 25° C. to about 55° C.

The step b) of aforementioned process involves the reaction of acyl-halamphetamine compound of general formula VI or isomer thereof, wherein X, R₄ and R₅ have the same meanings as identified above step a); with a silane reagent and a catalyst in presence of a suitable solvent under appropriate reaction conditions to provide acyl-amphetamine compound of general formula II or isomer thereof.

The silane reagent, catalyst and a suitable solvent used in the process is as defined above but also selected from triethylsilane, tetramethyldisiloxane, trichlorosilane, trimethylsilanol, phenylsilane, diphenylsilane, diphenylchlorosilane, tetraphenyldisilane and the like; preferably triethylsilane and tetramethyldisiloxane; the catalyst is selected from palladium hydroxide, palladium on carbon, platinum on carbon, Raney nickel, platinum oxide and the like; preferably palladium on carbon; and the suitable solvent is selected from water or alcohols such as methanol, ethanol, iso-propanol, n-butanol, iso-butanol and the like; preferably water.

The said reaction is carried out at a suitable temperature of about 0° C. to about 60° C. for sufficient period of time; preferably for about 4 to 5 hours at about 5° C. to about 35° C.

In another embodiment of the present invention provides an improved process for the preparation of acyl-amphetamine compound of general formula II or isomer thereof as shown in the below scheme:

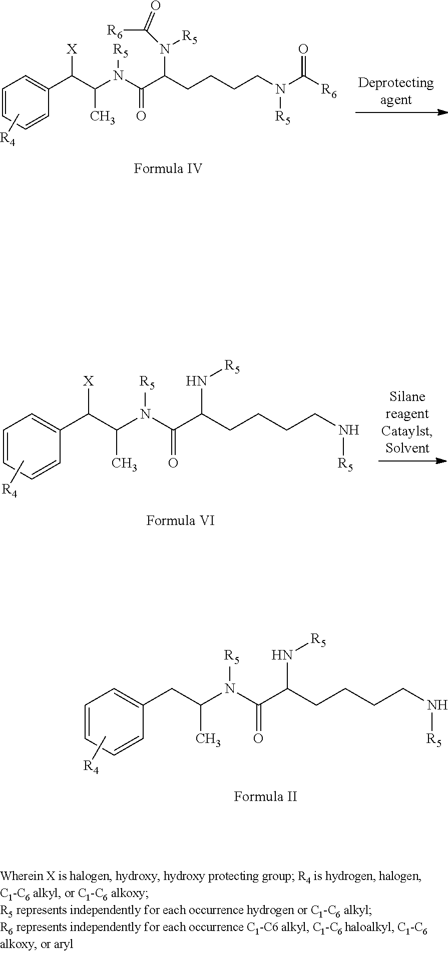

Certain compounds described herein may exist in particular geometric or stereoisomeric forms. The invention contemplates all such compounds, including cis- and trans-isomers, R- and S-enantiomers, diastereomers, (D)-isomers, (L)-isomers, the racemic mixtures thereof, and other mixtures thereof, as falling within the scope of the invention. Additional asymmetric carbon atoms may be present in a substituent such as an alkyl group. All such isomers, as well as mixtures thereof, are intended to be included in this invention. For generic chemical structures presented herein, the generic chemical structure is meant to encompass cis- and trans-isomers, a R-enantiomer, a S-enantiomer, diastereomers, and/or mixtures thereof unless the chemical structure or associated definition(s) species otherwise.

In the foregoing section, embodiments are described by way of examples to illustrate the processes of invention. However, these are not intended in any way to limit the scope of the present invention. Variants of the examples that would be evident to a person ordinarily skilled in the art are within the scope of the present invention.

### EXAMPLES

The process details of the invention are provided in the examples given below, which are provided by way of illustration only and therefore should not be construed to limit the scope of the invention.

### Example—1: Preparation of Amphetamine

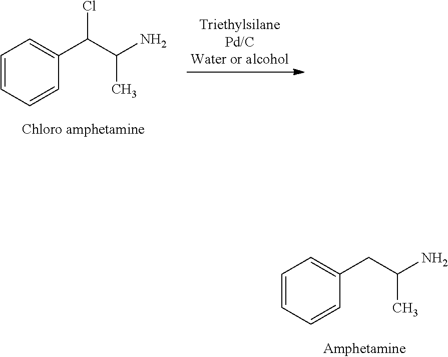

To a solution of Chloro-amphetamine in water or alcohol, Pd/C was added at room temperature. Triethylsilane was added to the reaction mass at room temperature and stirred for 1-3 hours at same temperature. After completion of the reaction, the reaction mass was filtered through hyflow and washed with water to get the titled compound.

### Example—2: Preparation of Dexamfetamine

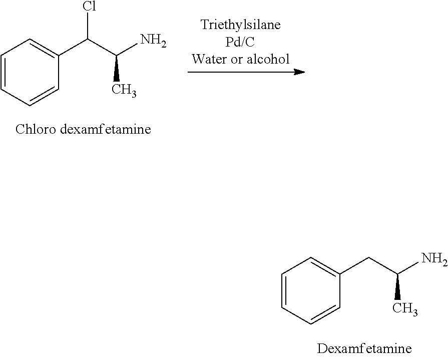

To a solution of Chloro-Dexamfetamine in water or alcohol, Pd/C was added at room temperature. Triethylsilane was added to the reaction mass at room temperature and stirred for 1-3 hours at same temperature. After completion of the reaction, the reaction mass was filtered through hyflow and washed with water to get the titled compound.

### Example—3: Preparation of N, N′-Bistrifluoroacetyl-Lisdexamfetamine

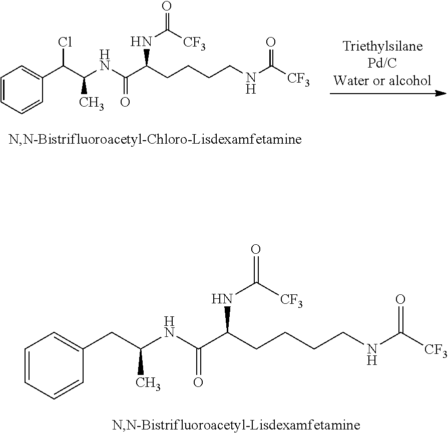 [SMILES: C[C@H](NC(=O)[C@H](CCCCNC(=O)C(F)(F)F)NC(=O)C(F)(F)F)C(Cl)c1ccccc1 ; C[C@@H](Cc1ccccc1)NC(=O)[C@H](CCCCNC(=O)C(F)(F)F)NC(=O)C(F)(F)F]

To a solution of N, N′-Bistrifluoroacetyl-Chloro-Lisdexamfetamine in water or alcohol, Pd/C was added at room temperature. Triethylsilane was added to the reaction mass at room temperature and stirred for 1-3 hours at same temperature. After completion of the reaction, the reaction mass was filtered through hyflow and washed 10 with water to get the titled compound.

### Example—4: Preparation of Lisdexamfetamine

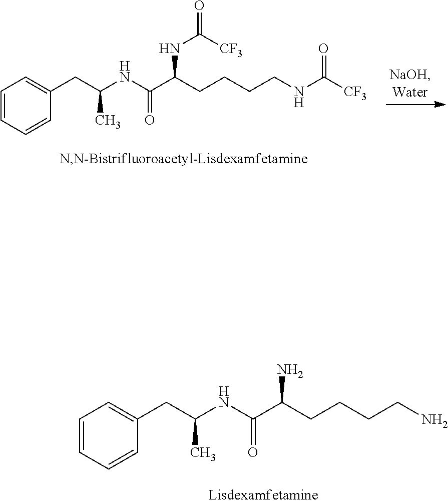 [SMILES: C[C@@H](Cc1ccccc1)NC(=O)[C@H](CCCCNC(=O)C(F)(F)F)NC(=O)C(F)(F)F ; C[C@@H](Cc1ccccc1)NC(=O)[C@@H](N)CCCCN]

Charged N, N′-Bistrifluoroacetyl-Lisdexamfetamine and 50% aqueous sodium hydroxide in water/alcohol at below 50° C. The mass was stirred at below 50° C. to get the titled compound.

### Example—5: Preparation of Chloro-Lisdexamfetamine

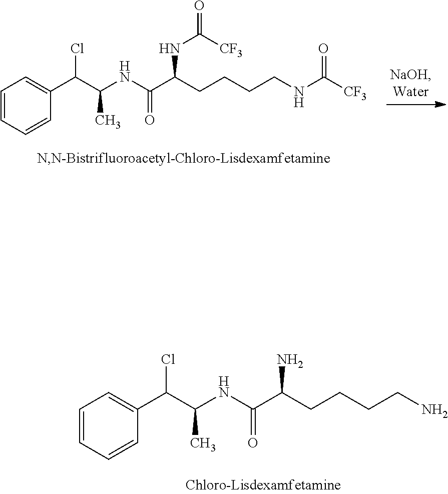 [SMILES: C[C@H](NC(=O)[C@H](CCCCNC(=O)C(F)(F)F)NC(=O)C(F)(F)F)C(Cl)c1ccccc1 ; C[C@H](NC(=O)[C@@H](N)CCCCN)C(Cl)c1ccccc1]

Charged N, N′-Bistrifluoroacetyl-Chloro-Lisdexamfetamine and 50% aqueous sodium hydroxide in water/alcohol at below 50° C. The mass was stirred at below 50° C. to get the titled compound.

### Example—6: Preparation of Lisdexamfetamine

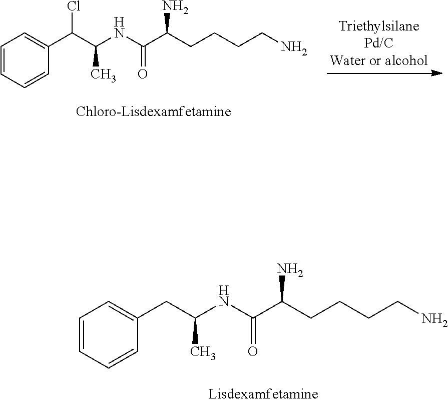 [SMILES: C[C@H](NC(=O)[C@@H](N)CCCCN)C(Cl)c1ccccc1 ; C[C@@H](Cc1ccccc1)NC(=O)[C@@H](N)CCCCN]

To a solution of Chloro-Lisdexamfetamine in water or alcohol, Pd/C was added at room temperature. Triethylsilane was added to the reaction mass at room temperature and stirred for 1-3 hours at same temperature. After completion of the reaction, the reaction mass was filtered through hyflow and washed with water to get the titled compound.

### Example—7: Di-Tert-Butoxycarbonyl-L-Lysine

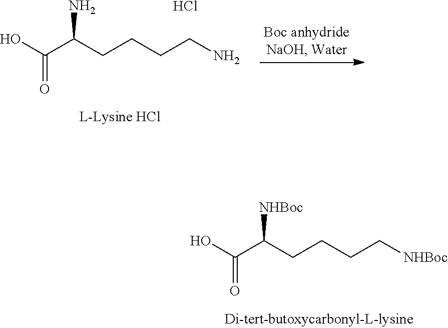 [SMILES: NCCCC[C@H](N)C(=O)O ; Cl ; CC(C)(C)OC(=O)NCCCC[C@H](NC(=O)OC(C)(C)C)C(=O)O]

Water (580 mL) and sodium hydroxide (38.1 g) and L-lysine HCl (58 g) were charged in a RB flask at 25 to 35° C., stirred for 5-10 min at the same temperature. Boc anhydride (173.2 g) was added to the reaction mass at 25 to 35° C. and stirred for 6 to 8 hours at the same temperature. n-Heptane (116 mL) was added to the reaction mass at 25 to 35° C., stirred for 10 to 15 min and settled for 10 to 20 min at same temperature. Aqueous and organic layers were separated; the pH of the aqueous layer was adjusted to 3.0-4.0 using aqueous citric acid solution at 25 to 35° C. and stirred for an hour at same temperature. MDC (348 mL) was added to the reaction mass at 25 to 35° C., stirred for 20 to 30 min and settled for 10 to 20 min at same temperature. Aqueous and MDC layers were separated, MDC (232 mL) was added into the aqueous layer at 25 to 35° C., stirred for 20 to 30 min and settled for 10 to 20 min at same temperature. Aqueous and MDC layers were separated, aqueous citric acid solution (8.7 g of citric acid in 165 mL of water) was added into the organic layer at 25 to 35° C., stirred for 20 to 30 min and settled for 10 to 20 min at same temperature. And this process of addition of aqueous citric acid solution repeated twice. Aqueous and MDC layers were separated, water (232 mL) was added to the MDC layer, stirred for 20 to 30 min and settled for 10 to 20 min at same temperature. Aqueous and MDC layers were separated, aqueous sodium chloride solution (58 g of sodium chloride in 232 mL of water) was added to the MDC layer, stirred for 10 to 20 min and settled for 10 to 20 min at same temperature. Aqueous and MDC layers were separated; the bottom MDC layer contains the product and proceeds to next step without isolation.

### Example—8: Preparation of N, N-Di-Boc-Lisdexamfetamine

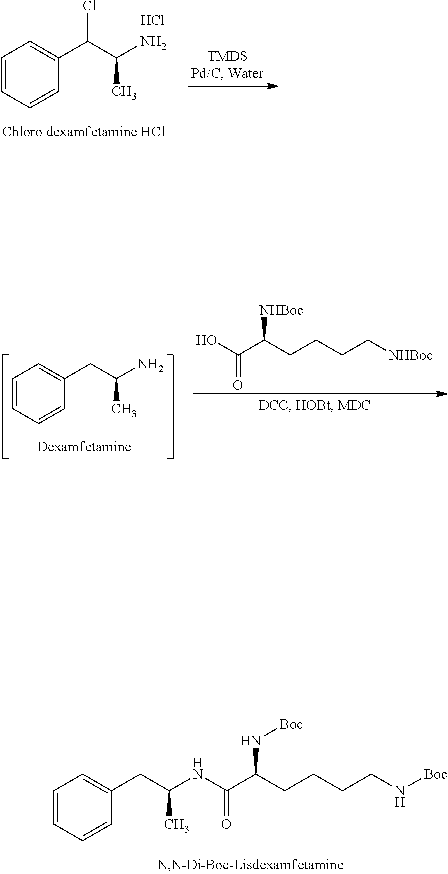

Chloro dexamfetamine hydrochloride (50 g) and water (300 mL) were charged into a RB flask, stirred for 10 to 15 min at 5 to 15° C., then 10% Pd/C (2.5 g) was added and stirred for 10 to 15 min at same temperature. Tetramethyldisiloxane (32.6 g) was added to the reaction mass at 5 to 15° C. and stirred for 4 to 5 hours at same temperature. The reaction mass was filtered through hyflow bed and washed twice with water. Toluene (150 mL) was added to the reaction mass at 25 to 35° C., stirred for 10 to 20 min and settled for 10 to 20 min at same temperature. Aqueous and organic layers were separated, toluene (150 mL) was added to the aqueous later 25 to 35° C., stirred for 10 to 20 min and settled for 10 to 20 min at same temperature. Aqueous and organic layers were separated, the pH of the aqueous layer adjusted to greater than 11 using aqueous sodium hydroxide solution at 25 to 35° C. and stirred for an hour at same temperature. MDC (250 mL) was added to the aqueous layer at 25 to 35° C., stirred for 10 to 20 min and settled for 10 to 20 min at same temperature. Aqueous and organic layers were separated, MDC (250 mL) was added to the aqueous layer at 25 to 35° C., stirred for 10 to 20 min and settled for 10 to 20 min at same temperature. Combine both the organic layers, water (250 mL) was added at 25 to 35° C., stirred for 10 to 20 min and settled for 10 to 20 min at same temperature. Aqueous and organic layers were separated, aqueous sodium chloride solution (20 g of sodium chloride in 200 mL of water) was added to the organic layer at 25 to 35° C., stirred for 10 to 20 min and settled for 10 to 20 min at same temperature. Aqueous and organic layers were separated; the bottom organic layer contains the product and proceeds to next step without isolation.

Di-tert-butoxycarbonyl-L-lysine in MDC (obtained from example 8), HOBT monohydrate (29.76 g) and DCC solution (76.68 g of DCC in 50 mL of MDC) were added to the above reaction mass at 0 to 10° C. and stirred for 4 to 6 hours at same temperature. Quench the reaction mass with water (250 mL) at below 30° C. and stirred for 4 to 6 hours at same temperature. The reaction mass was filtered, washed with MDC and settled for 20 to 30 min at 25 to 35° C. Aqueous and MDC layers were separated, aqueous sodium hydroxide solution (12.5 g of sodium hydroxide in 237.5 mL of water) was added to the MDC layer at 25 to 35° C., stirred for 20 to 30 min and settled for 30 to 40 min at same temperature. And this process of addition of aqueous sodium hydroxide solution repeated thrice. Combine all aqueous layers, MDC (150 mL) was added at 25 to 35° C., stirred for 20 to 30 min and settled for 30 to 40 min at same temperature. Combine all MDC layers, the MDC layer was extracted with aqueous HCl solution, followed by aqueous Sodium bicarbonate solution and aqueous Sodium chloride solution at 25 to 35° C., stirred for 10 to 20 min and settled for 30 to 40 min at same temperature. Distil-off the solvent at below 55° C., ethyl acetate (150 mL) was added to the reaction mass and distil-off the solvent under vacuum at below 60° C. Ethyl acetate (400 mL) was added to the reaction mass at below 60° C., cooled to 25 to 35° C., activated carbon (1 g) was added and stirred for an hour at same temperature. The reaction mass was filtered through hyflow bed, wash with ethyl acetate, distil-off the solvent under vacuum up at below 60° C. and stirred for 10 to 20 min at 50 to 60° C. n-Heptane (1 L) was added to the reaction mass 50 to 60° C., cooled to 25 to 35° C. and stirred for 2 to 3 hours at same temperature. The solid obtained was filtered, washed with n-heptane and dry for 30 min under vacuum. N-Heptane (600 mL) was added to the above obtained wet material, stirred for an hour at 25 to 35° C. The solid obtained was filtered, washed with n-heptane, suck dry for 30 min and dried for 6 to 8 hours at 50 to 60° C. under vacuum to get the titled compound. Yield: 83.20 g

### Example—9: Preparation of Lisdexamfetamine

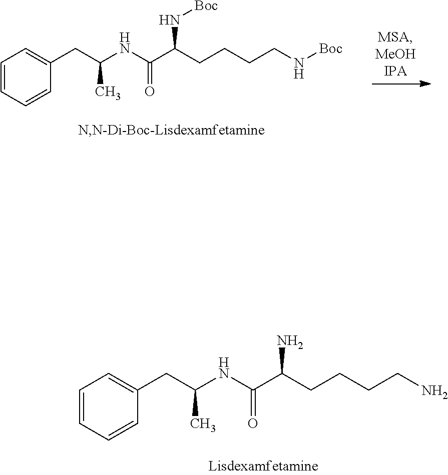 [SMILES: C[C@@H](Cc1ccccc1)NC(=O)[C@H](CCCCNC(=O)OC(C)(C)C)NC(=O)OC(C)(C)C ; C[C@@H](Cc1ccccc1)NC(=O)[C@@H](N)CCCCN]

N, N-Di-Boc-Lisdexamfetamine (100 g) and methanol (500 mL) were charged in a clean RB flask at 25 to 35° C., stirred for 10 to 20 min at same temperature. Methane sulfonic acid (51.8 g) was added to the reaction mass at 25 to 35° C., stirred for 10 to 20 min, temperature raised to 45 to 55° C. and stirred for 4 to 5 hours at same temperature. The reaction mass was filtered through micron filter, wash with pre-heated methanol and distil-off the solvent under vacuum at below 65° C. and stirred for 30 min at 45 to 55° C. Isopropyl alcohol (600 mL) was added into the reaction mass at 45 to 55° C., stirred for an hour, cooled to 25 to 35° C. and stirred for 1 to 2 hours at same temperature. The solid obtained was filtered, washed with isopropyl alcohol and dried under vacuum until no solvent expels out. Methanol (100 mL) was added into the reaction mass at 25 to 35° C., stirred for 10 to 20 min, temperature raised to 45 to 55° C. and stirred for 1 to 2 hours at same temperature. The reaction mass was cooled to 25 to 35° C., stirred for 6 to 8 hours, then isopropyl alcohol (600 mL) was added and stirred for 1 to 2 hours, again cooled to 15 to 25° C. and stirred for 2 to 3 hours at same temperature. The solid obtained was filtered, washed with isopropyl alcohol and dried under vacuum for 5 to 6 hours at 45 to 55° C. to get the titled compound. Yield: 88.4 g

We claim:

1. An improved process for the preparation of amphetamine compound of general formula I or isomer thereof: 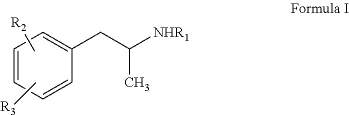
wherein R₁ is hydrogen or a lower alkyl group, and R₂ and R₃ are independently selected from hydrogen, halogen, lower alkyl groups, lower alkoxy groups, lower alkyl groups substituted with 1 to 5 halogens, lower alkoxy groups substituted with 1 to 5 halogens;
comprising reacting compound of general formula III or isomer thereof or its salt: 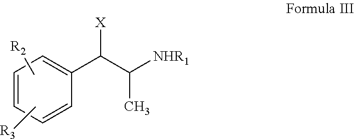
wherein X is halogen, hydroxy, hydroxy protecting group; R₁, R₂ and R₃ have the same meanings as identified above;
with a silane reagent and a catalyst in presence of a suitable solvent to provide amphetamine compound of general formula I or isomer thereof.

2. The process as claimed in claim 1, wherein the silane reagent is selected from triethylsilane, tetramethyldisiloxane, trichlorosilane, trimethylsilanol, phenylsilane, diphenylsilane, diphenylchlorosilane, tetraphenyldisilane, polymethylhydrosilane and polydimethylsiloxane.

3. The process as claimed in claim 1, wherein the catalyst is selected from palladium hydroxide, palladium on carbon, platinum on carbon, Fe, Raney nickel, platinum oxide and zinc; and the suitable solvent is selected from water or alcohols such as methanol, ethanol, iso-propanol, n-butanol and iso-butanol.

4. An improved process for the preparation of acyl-amphetamine compound of general formula II or isomer thereof: 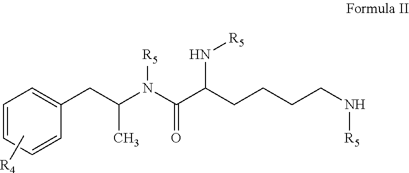
wherein R₄ is hydrogen, halogen, C₁-C₆ alkyl, or C₁-C₆ alkoxy; R₅ represents independently for each occurrence hydrogen or C₁-C₆ alkyl;
comprising:
a) reacting acyl-halamphetamine compound of general formula IV or isomer thereof: 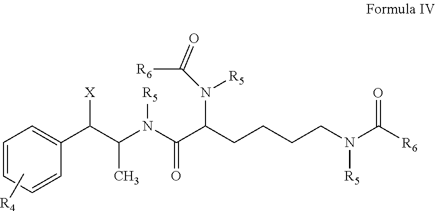
wherein X is halogen, hydroxy, hydroxy protecting group; R₄ and R₅ have the same meanings as identified above; R₆ represents independently for each occurrence C₁-C₆ alkyl, C₁-C₆ haloalkyl, C₁-C₆ alkoxy, or aryl;
with a silane reagent and a catalyst in presence of a suitable solvent to provide acyl-amphetamine compound of general formula V or isomer thereof; and 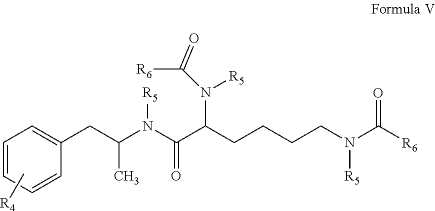
wherein R₄, R₅ and R₆ have the same meanings as identified above;
b) deprotecting the acyl-amphetamine compound of general formula V or isomer thereof with a suitable deprotecting agent to provide acyl-amphetamine compound of general formula II or isomer thereof.

5. The process as claimed in claim 4, wherein in step a) the silane reagent is selected from triethylsilane, tetramethyldisiloxane, trichlorosilane, trimethylsilanol, phenylsilane, diphenylsilane, diphenylchlorosilane, tetraphenyldisilane, polymethylhydrosilane and polydimethylsiloxane; the catalyst is selected from palladium hydroxide, palladium on carbon, platinum on carbon, Fe, Raney nickel, platinum oxide and zinc; and the suitable solvent is selected from water or alcohols such as methanol, ethanol, iso-propanol, n-butanol and iso-butanol.

6. The process as claimed in claim 4, wherein in step b) the suitable deprotecting agent is selected from hydrochloric acid, hydrobromic acid, sulfuric acid, methane sulfonic acid, p-toluene sulfonic acid, acetic acid, trifluoroacetic acid, lithium hydroxide, sodium hydroxide and potassium hydroxide.

7. An improved process for the preparation of acyl-amphetamine compound of general formula II or isomer thereof: 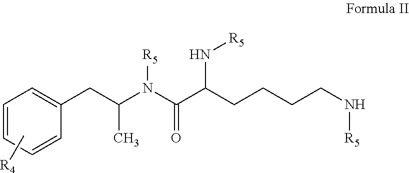
wherein R₄ is hydrogen, halogen, C₁-C₆ alkyl, or C₁-C₆ alkoxy; R₅ represents independently for each occurrence hydrogen or C₁-C₆ alkyl;
comprising:
a) deprotecting acyl-halamphetamine compound of general formula IV or isomer thereof: 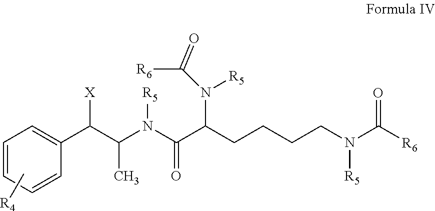
wherein X is halogen, hydroxy, hydroxy protecting group; R₄ and R₅ have the same meanings as identified above; R₆ represents independently for each occurrence C₁-C₆ alkyl, C₁-C₆ haloalkyl, C₁-C₆ alkoxy, or aryl;
with a suitable deprotecting agent to provide acyl-halamphetamine compound of general formula VI or isomer thereof; and 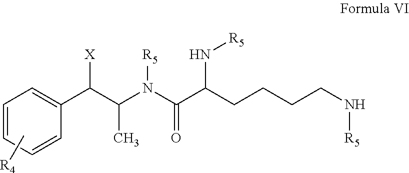
wherein X, R₄ and R₅ have the same meanings as identified above;
b) reacting the acyl-halamphetamine compound of general formula VI or isomer thereof with a silane reagent and a catalyst in presence of a suitable solvent to provide acyl-amphetamine compound of general formula II or isomer thereof.

8. The process as claimed in claim 7, wherein in step a) the suitable deprotecting agent is selected from hydrochloric acid, hydrobromic acid, sulfuric acid, methane sulfonic acid, p-toluene sulfonic acid, acetic acid, trifluoroacetic acid, lithium hydroxide, sodium hydroxide and potassium hydroxide.

9. The process as claimed in claim 7, wherein in step b) the silane reagent is selected from triethylsilane, tetramethyldisiloxane, trichlorosilane, trimethylsilanol, phenylsilane, diphenylsilane, diphenylchlorosilane, tetraphenyldisilane, polymethylhydrosilane and polydimethylsiloxane; the catalyst is selected from palladium hydroxide, palladium on carbon, platinum on carbon, Fe, Raney nickel, platinum oxide and zinc; and the suitable solvent is selected from water or alcohols such as methanol, ethanol, iso-propanol, n-butanol and iso-butanol.

10. An improved process for the preparation of Lisdexamphetamine comprising reacting compound of general formula III or isomer thereof or its derivative thereof or its salt: 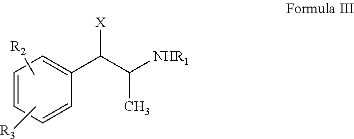
with a silane reagent as a hydrogen source and further converting it to Lisdexamphetamine.
